(19) United States
(12) Patent Application Publication
(10) Pub. No.: US 2021/0246497 A1
(43) Pub. Date: Aug. 12, 2021

(54) METHODS OF DETECTING NUCLEIC ACID

(71) Applicant: Glaxo Smith Kline Intellectual Property Development Limited, Brentford Middlesex (GB)

(72) Inventors: Shu Min ZHANG, Collegeville, PA (US); Matthew F. ROBERTS, Collegeville, PA (US)

(73) Assignee: Glaxo Smith Kline Intellectual Property Development Limited, Brentford Middlesex (GB)

(21) Appl. No.: 17/270,195

(22) PCT Filed: Aug. 26, 2019
(86) PCT No.: PCT/IB2019/057154

§ 371 Date: Feb. 22, 2021

Related U.S. Application Data
(60) Provisional application No. 62/724,696, filed on Aug. 30, 2018.

(51) Int. Cl. C12Q 1/6851; C12Q 1/6806

(52) U.S. Cl. CPC C12Q 1/6851; C12Q 1/6806

(57) ABSTRACT

The present invention relates to a method of detecting nucleic acid in a sample comprising a flocculant and a recombinant protein, the method comprising (a) adding heparin and a detergent to the sample, (b) amplifying at least a portion of the nucleic acid, and (c) detecting the amplification in step (b), thereby detecting the nucleic acid in the sample. The present invention also relates to a method of detecting nucleic acid in a sample comprising a flocculant and a recombinant protein, the method comprising (a) adding a detergent and sodium hydroxide to the sample, (b) amplifying at least a portion of the nucleic acid, and (c) detecting the amplification in step (b), thereby detecting the nucleic acid in the sample.

The present invention relates to a method of reducing interference in an assay for quantifying a nucleic acid in a recombinant protein sample by adding heparin and a detergent to the sample, or adding a detergent and sodium hydroxide to the sample, or adding to the sample a detergent and adjusting the pH of the sample to at least about 8.

### BACKGROUND OF THE INVENTION

Large-scale manufacture of recombinant proteins is an important challenge for the biotechnology industry. Recombinant proteins are usually produced by host cell culture or via cell free systems. In each case, the protein is purified from a sample comprising impurities to a purity sufficient for use as a human therapeutic product.

Typical processes involve initial clarification to remove solid particulates, followed by purification to ensure adequate purity. Clarification can lower the burden on subsequent chromatographic steps during purification. Typical clarification steps comprise a centrifugation step, or a filtration step, or both. Prior to clarification, a pre-treatment step may be used as a method of conditioning the sample. An example of a conditioning pre-treatment step is flocculation which causes solid particulates to form larger aggregates which are then removed by clarification. PEI (polyethyleneimine) is a flocculant and widely used in antibody purification processes.

During production of biopharmaceutical products, residual host cell DNA is an impurity that needs to be quantified to ensure it is within acceptable levels. The levels of residual DNA are typically closely monitored and controlled throughout the production process and release of the drug substance. Real-time quantitative PCR (qPCR) is a widely accepted approach for quantification of residual DNA in recombinant therapeutic proteins. However, residual PEI in samples (e.g., in-process or drug substance samples) strongly inhibits residual DNA qPCR assays and many other assays used in product quality and release testing for biopharmaceutical products. Typically, very high sample dilutions (e.g., 1:10,000) are required to overcome assay interference due to the presence of PEI in concentrations greater than or equal to 20 ppm. Diluting samples this much poses a significant risk to the assay sensitivity needed to satisfy regulatory requirements regarding the amount of residual host cell genomic DNA present per parenteral dose (e.g., 10 ng/dose).

Thus, a need exists for methods for improving assay sensitivity in the quantification of residual host cell DNA in recombinant protein samples.

### SUMMARY OF THE INVENTION

In one aspect, a method for detecting a nucleic acid in a sample comprising a recombinant protein and a flocculant is provided, the method comprising
(a) adding a detergent and sodium hydroxide (NaOH) to the sample,
(b) amplifying at least a portion of the nucleic acid, and
(c) detecting the amplification in step (b), thereby detecting the nucleic acid in the sample.

In another aspect, a method for detecting a nucleic acid in a sample comprising a recombinant protein and a flocculant is provided, the method comprising
(a) adding a detergent to the sample,
(b) adjusting the pH of the sample to at least about 8,
(c) amplifying at least a portion of the nucleic acid, and
(d) detecting the amplification in step (c), thereby detecting the nucleic acid in the sample.

In still another aspect, a method for detecting nucleic acid in a sample comprising a flocculant and a recombinant protein is provided, the method comprising
(a) adding heparin and a detergent to the sample,
(b) amplifying at least a portion of the nucleic acid, and
(c) detecting the amplification in step (b), thereby detecting the nucleic acid in the sample.

### BRIEF DESCRIPTION OF THE FIGURES

FIG. 1A is a schematic showing the molecular structure of polyethyleneimine (PEI); a repeating unit of PEI (top) and an example branched PEI fragment (bottom) are shown.

FIG. 1B is a schematic showing PEI binding DNA and forming a complex. FIG. 1C is a schematic showing the molecular structure of heparin.

FIG. 2 is a table showing recovery in samples obtained under different conditions, such as process conditions using different wash buffers and elution buffers.

FIG. 3 is a schematic showing the experiment procedure for determining the effect of adding heparin and sarkosyl to samples containing PEI and DNA on assay sensitivity.

FIG. 4A is a table showing spiking recovery in samples treated with heparin and sarkosyl. FIG. 4B is a plot of the results shown in the table in FIG. 4A.

FIG. 5 is a set of tables and plots showing spiking recovery in mAb 1 eluate with 100 ppm PEI treated with heparin (80 μg/mL) and sarkosyl (0.05%).

FIG. 6 is a plot showing spiking recovery in mAb1 bulk drug substance (BDS) samples with 20 ppm PEI treated with Sarkosyl or SDS plus NaOH.

FIG. 7 is a plot showing spiking recovery in mAb2 bulk drug substance (BDS) samples with 20 ppm PEI and 10⁴ μg/mL Chinese hamster ovary (CHO) DNA treated with different concentration of SDS and NaOH.

FIG. 8 is a set of plots showing spiking recovery in mAb2 bulk drug substance (BDS) samples with 20 ppm PEI at varying concentrations of NaOH.

FIG. 9 is a set of plots showing spiking recovery in mAb1mAb2, and mAb3 bulk drug substance (BDS) samples with 20 ppm PEI and 10⁴ μg/mL Chinese hamster ovary (CHO) DNA treated with 0.5% SDS and 25 mM NaOH, and by Wako DNA extraction.

### DETAILED DESCRIPTION

The present invention is based, at least in part, on the discovery that a more sensitive assay for quantifying residual DNA in a recombinant protein sample (e.g., in-process or bulk drug substance samples) containing PEI can be achieved by treating the sample with heparin and a detergent (e.g., sarkosyl), or a detergent (e.g., SDS) and sodium hydroxide, prior to performing qPCR analysis. Trace amounts of PEI in a sample can inhibit a number of assays, in particular, residual host cell DNA qPCR.

Removing PEI and retaining DNA, overcoming the interference of polyethyleneimine (PEI) on the residual host cell DNA qPCR assay and other assays used for process development and for drug substance release testing is very important for achieving the assay sensitivity necessary to demonstrate DNA clearance for biopharmaceutical products.

Without intending to be bound by theory, treating the sample with heparin and a detergent or with a detergent and sodium hydroxide is believed to reduce the interaction between PEI and DNA, therefore resulting in reduced interference from PEI and improved qPCR assay sensitivity. Without intending to be bound by theory, it is believed that PEI binds DNA, and that negatively charged molecules (e.g., heparin) can competitively bind to PEI (PEI is positively charged) and thereby release the DNA.

It is to be understood that this invention is not limited to particular methods, reagents, compounds, compositions, or biological systems, which can, of course, vary. It is also to be understood that the terminology used herein is not intended to be limiting but is used for the purpose of describing particular embodiments only. As used in this specification and the appended claims, the singular forms “a”, “an”, and “the” include plural referents unless the content clearly dictates otherwise. Thus, for example, reference to “a polypeptide” includes a combination of two or more polypeptides, and the like.

The term “comprising” encompasses “including” or “consisting” e.g. a composition “comprising” X may consist exclusively of X or may include something additional e.g. X+Y. The term “consisting essentially of” limits the scope of the feature to the specified materials or steps and those that do not materially affect the basic characteristic(s) of the claimed feature. The term “consisting of” excludes the presence of any additional component(s).

“About” as used herein when referring to a measurable value such as an amount, a temporal duration, and the like, is meant to encompass variations of ±20% or ±10%, including ±5%, ±1%, and ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### Recombinant Protein

“Polypeptide,” “peptide” and “protein” are used interchangeably herein to refer to a polymer of amino acid residues.

“Peptide” as used herein includes peptides which are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acids, or glutamine for asparagine, and the like. Neutral hydrophilic amino acids which can be substituted for one another include asparagine, glutamine, serine and threonine.

“Conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the proteins described herein.

As used herein a “therapeutic protein” refers to any protein and/or polypeptide that can be administered to a mammal to elicit a biological or medical response of a tissue, system, animal or human. The recombinant protein may elicit more than one biological or medical response. Furthermore, the term “therapeutically effective amount” means any amount which, as compared to a corresponding subject who has not received such amount, results in, but is not limited to, healing, prevention, or amelioration of a disease, disorder, or side effect, or a decrease in the rate of advancement of a disease or disorder. The term also includes within its scope amounts effective to enhance normal physiological function as well as amounts effective to cause a physiological function in a patient which enhances or aids in the therapeutic effect of a second pharmaceutical agent.

“Recombinant” when used with reference to a protein indicates that the protein has been recombinantly expressed in a host cell.

The recombinant protein may comprise an antigen binding protein, for example an antibody, a monoclonal antibody, an antibody fragment, or a domain antibody. The recombinant protein may comprise a viral protein, a bacterial toxin, a bacterial toxoid, or a cancer antigen. In one embodiment, the recombinant protein is a monoclonal antibody.

The term “antigen binding protein” as used herein refers to antibodies, antibody fragments and other protein constructs, such as domains, which are capable of binding to an antigen.

The term “antibody” is used herein in the broadest sense to refer to molecules with an immunoglobulin-like domain. As used herein, “immunoglobulin-like domain” refers to a family of polypeptides which retain the immunoglobulin fold characteristic of antibody molecules, which contain two β-sheets and, usually, a conserved disulphide bond. This family includes monoclonal (for example IgG, IgM, IgA, IgD or IgE), recombinant, polyclonal, chimeric, humanised, bispecific and heteroconjugate antibodies; a single variable domain, a domain antibody, antigen binding fragments, immunologically effective fragments, Fab, F(ab′)₂, Fv, disulphide linked Fv, single chain Fv, diabodies, TANDABS™, etc.

The phrase “single variable domain” refers to an antigen binding protein variable domain (for example, V_{H}, V_{HH}, V_{L}) that specifically binds an antigen or epitope independently of a different variable region or domain. A “domain antibody” or “dAb” may be considered the same as a “single variable domain” which is capable of binding to an antigen or epitope. The term “epitope-binding domain” refers to a domain that specifically binds an antigen or epitope independently of a different domain.

A domain antibody can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains).

The domain antibody may be a human antibody variable domain. The dAb may be of human origin. In other words, the dAb may be based on a human Ig framework sequence.

As used herein, the term “antigen binding site” refers to a site on an antigen binding protein which is capable of specifically binding to an antigen, this may be a single domain, or it may be paired V_{H}/V_{L}. domains as can be found on a standard antibody. Single-chain Fv (ScFv) domains can also provide antigen-binding sites.

The antigen binding protein may take the protein scaffold format of a mAbdAb. “mAbdAb” and “dAbmAb” are used interchangeably and are intended to have the same meaning as used herein. Such antigen-binding proteins comprise a protein scaffold, for example an Ig scaffold such as IgG, for example a monoclonal antibody, which is linked to a further binding domain, for example a domain antibody. A mAbdAb has at least two antigen binding sites, at least one of which is from a domain antibody, and at least one is from a paired V_{H}/V_{L}. domain.

As used herein, “drug” refers to any compound (for example, a small organic molecule, a nucleic acid, a polypeptide) that can be administered to an individual to produce a beneficial therapeutic or diagnostic effect through binding to and/or altering the function of a biological target molecule in the individual. The target molecule can be an endogenous target molecule encoded by the individual's genome (e.g., an enzyme, receptor, growth factor, cytokine encoded by the individual's genome) or an exogenous target molecule encoded by the genome of a pathogen. The drug may be a dAb or mAb.

A “dAb conjugate” refers to a composition comprising a dAb to which a drug is chemically conjugated by means of a covalent or noncovalent linkage. Preferably, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin), wherein one partner is covalently bonded to drug and the complementary binding partner is covalently bonded to the dAb). When complementary binding partners are employed, one of the binding partners can be covalently bonded to the drug directly or through a suitable linker moiety, and the complementary binding partner can be covalently bonded to the dAb directly or through a suitable linker moiety.

As used herein, “dAb fusion” refers to a fusion protein that comprises a dAb and a polypeptide drug (which could be a polypeptide, a dAb or a mAb). The dAb and the polypeptide drug are present as discrete parts (moieties) of a single continuous polypeptide chain.

Methods of the disclosure may be applied to detect nucleic acid in samples containing one or more of: a recombinant protein, an antigen binding protein, an antibody, a monoclonal antibody (mAb), a domain antibody (dAb), a dAb conjugate, a dAb fusion, a mAbdAb, or any other antigen binding protein described above.

In one embodiment, the recombinant protein sample comprises a therapeutic protein. In another embodiment, the sample comprises an antigen binding protein. In one embodiment, the sample comprises a monoclonal antibody.

### Expression of Protein

The recombinant protein may be prepared by any of a number of conventional techniques. For example, the protein may be purified from cells that naturally express them (e.g., an antibody can be purified from a hybridoma that produces it), or produced in a recombinant expression system. In one embodiment, the recombinant proteins are produced/derived from a mammalian cell or a bacterial cell. In a further embodiment the mammalian cell is selected from a human or rodent (such as a hamster or mouse) cell. In a yet further embodiment the human cell is a HEK cell, the hamster cell is a CHO cell or the mouse cell is a NSO cell. In one embodiment, the host cell is a CHO cell.

In certain embodiments the host cell is selected from the group consisting of selected from the group consisting of CHO cells, NSO cells, Sp2/0 cells, COS cells, K562 cells, BHK cells, PER.C6 cells, and HEK cells. Alternatively, the host cell may be a bacterial cell selected from the group consisting of E. coli (for example, W3110, BL21), B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa).

A vector comprising a recombinant nucleic acid molecule encoding the recombinant protein is also described herein. The vector may be an expression vector comprising one or more expression control elements or sequences that are operably linked to the recombinant nucleic acid. Examples of vectors include plasmids and phagemids.

Suitable expression vectors can contain a number of components, for example, an origin of replication, a selectable marker gene, one or more expression control elements, such as a transcription control element (e.g. promoter, enhancer, terminator) and/or one or more translation signals, a signal sequence or leader sequence. Expression control elements and a signal sequence, if present, can be provided by the vector or other source. For example, the transcriptional and/or translational control sequences of a cloned nucleic acid encoding an antibody chain can be used to direct expression. A promoter can be provided for expression in a desired cell. Promoters can be constitutive or inducible. For example, a promoter can be operably linked to a nucleic acid encoding an antibody, antibody chain or portion thereof, such that it directs transcription of the nucleic acid.

The host cell comprises the recombinant nucleic acid molecule or vector described above. The recombinant protein may be expressed intracellularly. In another embodiment, the expressed recombinant protein has a signal sequence (also known as a signal peptide), which routes the protein along the secretory pathway of the cell.

The host cell is grown under conditions suitable for expression of the recombinant protein. Host cell cultures may be cultured in any medium that supports the host cell growth and expression of the recombinant protein. Such media are well known to those skilled in the art. Although expression of a recombinant protein takes place in the cytoplasm of the host cell, the final location of the recombinant protein may be cytoplasmic, periplasmic or extracellular depending on the nature of the recombinant protein, the host cell used, and the fermentation conditions used.

The fermentor volume may be:

(i) about 10,000 litres; about 5,000 litres; about 2,000 litres; about 1,000 litres; about 500 litres; about 125 litres; about 50 litres; about 20 litres; about 10 litres; about 5 litres; or

(ii) between 5 and 10,000 litres; between 10 and 5,000 litres; between 20 and 2,000 litres; between 50 and 1,000 litres.

Harvest is the end of fermentation. Harvest may be at any time point during fermentation that is considered sufficient to end the fermentation process and recover the recombinant protein being expressed. Harvest may include the optional step of emptying the fermentor of the cells and extracellular media (i.e. the cell culture or broth).

### Purification of Protein

Typical purification processes involve initial clarification to remove solid particulates, followed by purification to ensure adequate purity of the recombinant protein.

Clarification can lower the burden on subsequent chromatographic steps during purification. Typical clarification steps comprise a settling step—also known as sedimentation (e.g. by gravity), and/or a centrifugation step, and/or a filtration step. Prior to clarification, a pre-treatment step may be used as a method of conditioning the sample. An example of a conditioning pre-treatment step is flocculation which causes solid particulates to form larger aggregates which are then removed by clarification.

One or more chromatography steps may be used in purification, for example one or more chromatography resins; and/or one or more filtration steps. For example, affinity chromatography using resins such as protein A or L may be used to purify the recombinant protein. Alternatively, or in addition to, an ion-exchange resin such as a cation-exchange resin may be used to purify the recombinant protein.

The purified recombinant protein may be formulated in a pharmaceutically acceptable composition.

### Sample

As used herein, a “bulk drug substance” sample or “BDS” sample is a sample that contains a high concentration of recombinant protein. Typically, a bulk drug substance sample has a protein concentration of about 50 mg/mL to about 250 mg/mL. In one embodiment, the bulk drug substance sample has a protein concentration of about 100 mg/mL to about 120 mg/mL.

In one embodiment, the BDS sample comprises at least about 50 mg/mL recombinant protein, at least about 100 mg/mL recombinant protein, at least about 105 mg/mL recombinant protein, at least about 110 mg/mL recombinant protein, at least about 115 mg/mL recombinant protein, at least about 120 mg/mL recombinant protein, at least about 125 mg/mL recombinant protein, at least about 150 mg/mL recombinant protein, at least about 200 mg/mL recombinant protein, or at least about 250 mg/mL recombinant protein. In one embodiment, the BDS sample comprises about 50 mg/mL to about 250 mg/mL recombinant protein. In another embodiment, the BDS sample comprises about 100 mg/mL to about 200 mg/mL recombinant protein. In another embodiment, the BDS sample comprises about 100 mg/mL to about 150 mg/mL recombinant protein. In another embodiment, the BDS sample comprises about 100 mg/mL to about 120 mg/mL recombinant protein. In one embodiment, the recombinant protein is a monoclonal antibody.

As used herein, an “in-process” sample is a sample that contains a low concentration of recombinant protein. For example, an in-process sample has a protein concentration typically has a protein concentration of about 0.1 mg/mL to about 20 mg/mL. In one embodiment, the in-process sample has a protein concentration of about 1 mg/mL to about 15 mg/mL.

In one embodiment, the in-process sample comprises about 0.1 mg/mL recombinant protein to about 20 mg/mL. In one embodiment, the sample in-process comprises about 0.5 mg/mL to about 20 mg/mL recombinant protein. In another embodiment, the in-process sample comprises about 1 mg/mL to about 20 mg/mL recombinant protein. In another embodiment, the in-process sample comprises about 1 mg/mL to about 15 mg/mL recombinant protein. In one embodiment, the recombinant protein is a monoclonal antibody.

### Analytical Methods

The methods described herein can be used to remove polyethyleneimine (PEI) from a sample, thereby improving assay sensitivity in a variety of analytical methods. Such analytical methods include, but are not limited to, real time quantitative PCR (qPCR), capillary gel electrophoresis (CGE), surface plasmon resonance (e.g., BiacoreTM), and reverse phase HPLC.

There is a provided a method of reducing interference and/or increasing sensitivity in an assay for detecting an analyte in a sample comprising a flocculant, the method comprising adding heparin and a detergent to the sample, thereby reducing interference and/or increasing sensitivity in the assay for detecting the analyte in the sample. In one embodiment, the flocculant is PEI. In one embodiment, the analyte is a nucleic acid. In one embodiment, the sample further comprises a recombinant protein. In one embodiment, the detergent is sarkosyl or SDS. In one embodiment, the assay is a biopharmaceutical analytical method. In another embodiment, the assay is an amplification-based assay for detecting nucleic acid. In one embodiment, the assay is qPCR. In another embodiment, the assay is capillary gel electrophoresis (CGE). In another embodiment, the assay is surface plasmon resonance. In another embodiment, the assay is reverse phase HPLC. In one embodiment, heparin is added to the sample to a final concentration of about 50 μg/mL to about 1000 μg/mL. In one embodiment, sarkosyl is added to the sample to a final concentration of about 0.01% to about 2.0%.

There is provided a method for reducing interference and/or increasing sensitivity in an assay for detecting an analyte in a sample comprising a flocculant, the method comprising (a) adding to the sample detergent and sodium hydroxide (NaOH), thereby reducing interference and/or increasing sensitivity in the assay for detecting the analyte in the sample. In one embodiment, the flocculant is PEI. In one embodiment, the analyte is a nucleic acid. In one embodiment, the sample further comprises a recombinant protein. In one embodiment, the detergent is sarkosyl or SDS. In one embodiment, the assay is a biopharmaceutical analytical method. In another embodiment, the assay is an amplification-based assay for detecting nucleic acid. In one embodiment, the assay is qPCR. In another embodiment, the assay is capillary gel electrophoresis (CGE). In another embodiment, the assay is surface plasmon resonance. In another embodiment, the assay is reverse phase HPLC. In one embodiment, SDS is added to the sample to a final concentration of about 0.01% to about 2.0%. In one embodiment, NaOH is added to the sample to a final concentration of about 0.1 mM to about 100 mM.

There is provided a method for reducing interference and/or increasing sensitivity in an assay for detecting an analyte in a sample comprising a flocculant, the method comprising (a) adding to the sample with a detergent, and (b) adjusting the pH of the sample to at least about 8, thereby reducing interference and/or increasing sensitivity in the assay for detecting the analyte in the sample. In one embodiment, the flocculant is PEI. In one embodiment, the analyte is a nucleic acid. In one embodiment, the sample further comprises a recombinant protein. In one embodiment, the detergent is sarkosyl or SDS. In one embodiment, the assay is a biopharmaceutical analytical method. In another embodiment, the assay is an amplification-based assay for detecting nucleic acid. In one embodiment, the assay is qPCR. In another embodiment, the assay is capillary gel electrophoresis (CGE). In another embodiment, the assay is surface plasmon resonance. In another embodiment, the assay is reverse phase HPLC. In one embodiment, the pH is adjusted to about 9. In some embodiments, SDS is added to the sample to a final concentration of about 0.01% to 2.0%. In one aspect, there is provided a method for detecting a nucleic acid in a sample comprising a recombinant protein and a flocculant, the method comprising (a) adding a detergent and sodium hydroxide (NaOH) to the sample; (b) amplifying at least a portion of the nucleic acid; and (c) detecting the amplification in step (b), thereby detecting the nucleic acid in the sample.

In one aspect, there is provided a method of reducing interference and/or increasing sensitivity in an assay for detecting a nucleic acid in a sample comprising a flocculant and a recombinant protein, the method comprising (a) adding heparin and a detergent to the sample, wherein step (a) reduces the interaction between the nucleic acid and flocculant that inhibits amplification of the nucleic acid during the assay, thereby reducing interference and/or increasing sensitivity in the assay for detecting nucleic acid in the sample. In one aspect, there is provided a method for detecting a nucleic acid in a sample comprising a recombinant protein and a flocculant, the method comprising (a) adding a heparin and a detergent to the sample; (b) amplifying at least a portion of the nucleic acid; and (c) detecting the amplification in step (b), thereby detecting the nucleic acid in the sample; wherein step (a) reduces the interaction between the nucleic acid and flocculant that inhibits amplification of the nucleic acid during the assay, thereby reducing interference and/or increasing sensitivity in the assay for detecting nucleic acid in the sample. In some embodiments, the assay is qPCR. In one embodiment, heparin is added to the sample to a final concentration of about 50 μg/mL to about 1000 μg/mL. In one embodiment, the detergent is sarkosyl. In one embodiment, sarkosyl is added to the sample to a final concentration of about 0.01% to about 2.0%.

In one aspect, there is provided a method for detecting a nucleic acid using qPCR in a sample comprising a recombinant protein and a flocculant, the method comprising (a) adding a heparin and a detergent to the sample; (b) amplifying at least a portion of the nucleic acid; and (c) detecting the amplification in step (b), thereby detecting the nucleic acid in the sample; wherein step (a) reduces the interaction between the nucleic acid and flocculant that inhibits amplification of the nucleic acid during the assay, thereby reducing interference and/or increasing sensitivity in the assay for detecting nucleic acid in the sample.

In one aspect, there is provided a method for detecting a nucleic acid using qPCR in a sample comprising a recombinant protein and a flocculant, the method comprising (a) adding a heparin and a detergent to the sample, wherein heparin is added to the sample to a final concentration of about 50 μg/mL to about 1000 μg/mL; (b) amplifying at least a portion of the nucleic acid; and (c) detecting the amplification in step (b), thereby detecting the nucleic acid in the sample; wherein step (a) reduces the interaction between the nucleic acid and flocculant that inhibits amplification of the nucleic acid during the assay, thereby reducing interference and/or increasing sensitivity in the assay for detecting nucleic acid in the sample.

In one aspect, there is provided a method for detecting a nucleic acid using qPCR in a sample comprising a recombinant protein and a flocculant, the method comprising (a) adding a heparin and a detergent to the sample, wherein the detergent is sarkosyl, and added to the sample to a final concentration of about 0.01% to about 2%; (b) amplifying at least a portion of the nucleic acid; and (c) detecting the amplification in step (b), thereby detecting the nucleic acid in the sample; wherein step (a) reduces the interaction between the nucleic acid and flocculant that inhibits amplification of the nucleic acid during the assay, thereby reducing interference and/or increasing sensitivity in the assay for detecting nucleic acid in the sample.

In one aspect, there is provided a method for detecting a nucleic acid using qPCR in a sample comprising a recombinant protein and a flocculant, the method comprising (a) adding a heparin and a detergent to the sample, wherein the detergent is SDS, and added to the sample to a final concentration of about 0.01% to about 2%; (b) amplifying at least a portion of the nucleic acid; and (c) detecting the amplification in step (b), thereby detecting the nucleic acid in the sample; wherein step (a) reduces the interaction between the nucleic acid and flocculant that inhibits amplification of the nucleic acid during the assay, thereby reducing interference and/or increasing sensitivity in the assay for detecting nucleic acid in the sample.

In another aspect, there is provided a method for reducing interference and/or increasing sensitivity in an amplification-based assay for detecting nucleic acid in a sample comprising a recombinant protein and a flocculant, the method comprising adding to the sample a detergent and sodium hydroxide (NaOH), wherein step (a) reduces nucleic acid-flocculant interaction that inhibits amplification of the nucleic acid during the assay, thereby reducing interference and/or increasing sensitivity in the assay for detecting nucleic acid in the sample. In one embodiment, the assay is qPCR. In one embodiment, the detergent is SDS. In one embodiment, SDS is added to the sample to a final concentration of about 0.01% to about 2.0%. In one embodiment, NaOH is added to the sample to a final concentration of about 0.1 mM to about 100 mM.

In still another aspect, there is provided a method for reducing interference and/or increasing sensitivity in an amplification-based assay for detecting nucleic acid in a sample comprising a recombinant protein and a flocculant, the method comprising (a) adding a detergent to the sample, and (b) adjusting the pH of the sample to at least about 8, wherein steps (a) and (b) reduce nucleic acid-flocculant interaction that inhibits amplification of the nucleic acid during the assay, thereby reducing interference and/or increasing sensitivity in the assay for detecting nucleic acid in the sample. In one embodiment, the assay is qPCR. In one embodiment, the pH is adjusted to about 9. In one embodiment, SDS is added to the sample to a final concentration of about 0.01% to 2.0%.

In one embodiment, treatment of the sample according to the method results in increased sensitivity of the assay of about 5-fold to 200-fold when compared to sensitivity of the assay using an untreated sample. In one embodiment, the sensitivity of the assay is increased about 5-fold, about 10-fold, about 15-fold, about 20-fold, about 30-fold, about 40-fold, about 50-fold, about 60-fold, about 70-fold, about 80-fold, about 90-fold, about 100-fold, about 120-fold, about 140-fold, about 160-fold, about 180-fold, or about 200-fold, when compared to sensitivity of the assay using an untreated sample.

In one embodiment, the assay is an amplification-based assay for detecting nucleic acid. In one embodiment, the assay is qPCR. In one embodiment, the treatment of the sample according to the method results in acceptable recovery when the sample is diluted at a dilution factor of about 1:200 to about 1:500, compared to a dilution factor for about 1:2000 to 1:10000 when using an untreated sample.

### Methods for Detecting or Quantifying Nucleic Acids

The polymerase chain reaction (PCR) is a common thermal cycling dependent nucleic acid amplification technology used to amplify DNA consisting of cycles of repeated heating and cooling of the reaction for DNA melting and enzymatic replication of the DNA using a DNA polymerase. Real-Time quantitative PCR (qPCR) is a technique used to quantify the number of copies of a given nucleic acid sequence in a biological sample. Currently, qPCR utilizes the detection of reaction products in real-time throughout the reaction and compares the amplification profile to the amplification of controls which contain a known quantity of nucleic acids at the beginning of each reaction (or a known relative ratio of nucleic acids to the unknown tested nucleic acid). The results of the controls are used to construct standard curves, typically based on the logarithmic portion of the standard reaction amplification curves. These values are used to interpolate the quantity of the unknowns based on where their amplification curves compared to the standard control quantities.

In addition to PCR, non-thermal cycling dependent amplification systems or isothermal nucleic acid amplification technologies exist including, without limitation: Nicking Amplification Reaction, Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-β Replicase System, and Recombinase Polymerase Amplification (RPA). Other amplification technologies include ligase chain reaction (LCR), multiple displacement amplification (MDA), helicase dependent amplification (HDA), and ramification dependent amplification (RAM).

The TaqMan qPCR system is widely used for qualification of residual host cell DNA. The TaqMan qPCR system add a fluorescence labelled probe between two primers, during PCR synthesize DNA fragments the polymerase clip probe, reporter dye lost control from quencher dye and generate fluorescence signals proportionally with the number of DNA fragments synthesized by PCR. The 96 well format with high throughput dramatically increased efficiency and quantification power than first generation of PCR, conventional PCR. qPCR method is excellent in terms of sensitivity and specificity but vulnerable to the interference from sample matrices. Normally residual DNA host cell DNA qPCR assay require sample dilution, DNA extraction and spiking positive control DNA and assess spiking recovery.

In one embodiment, nucleic acid in a sample is detected or quantified via real-time quantitative PCR (qPCR). In one embodiment, host cell DNA in a sample is detected or quantified via real-time quantitative PCR (qPCR).

### Detergent

A detergent is added to a recombinant protein sample. In one embodiment, the detergent is sarkosyl. Sarkosyl is an anionic surfactant. Sarkosyl can be used for cell lysis and protein solubilization. The structure of sarkosyl is provided below:

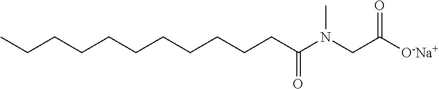

In another embodiment, the detergent is sodium dodecyl sulfate (SDS). The structure of SDS is provided below:

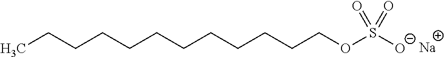 [SMILES: CCCCCCCCCCCCOS(=O)(=O)[O-] ; [Na+]]

In some other embodiments, the detergent is sodium cholate, sodium deoxycholate, Triton X 100, or Tween 20.

In one embodiment, detergent is added to a recombinant protein sample so that the concentration of detergent in the sample is about 0.01% to about 2.0%. In another embodiment, the concentration of detergent in the sample is about 0.05% to about 1.0%. In one embodiment, the concentration of the detergent in the sample is at least about 0.01%, at least about 0.05%, at least about 0.1%, at least about 0.5%, at least about 1.0%, at least about 1.5%, or at least about 2.0%. In another embodiment, the concentration of the detergent in the sample is about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9% or about 2.0%.

In one embodiment, sarkosyl is added to a recombinant protein sample so that the concentration of sarkosyl in the sample is about 0.01% to about 2.0%. In another embodiment, the concentration of sarkosyl in the sample is about 0.05% to about 1.0%. In one embodiment, the concentration of the sarkosyl in the sample is at least about 0.01%, at least about 0.05%, at least about 0.1%, at least about 0.5%, at least about 1.0%, at least about 1.5%, or at least about 2.0%. In another embodiment, the concentration of the sarkosyl in the sample is about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In one embodiment, sodium dodecyl sulfate (SDS) is added to a recombinant protein sample so that the concentration of SDS in the sample is about 0.01% to 2.0%. In another embodiment, the concentration of SDS in the sample is about 0.05% to about 1.0%. In one embodiment, the concentration of the SDS in the sample is at least about 0.01%, at least about 0.05%, at least about 0.1%, at least about 0.5%, at least about 1.0%, at least about 1.5%, or at least about 2.0%. In another embodiment, the concentration of the SDS in the sample is about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In one embodiment, the pH of the sample is at least about 8. In another embodiment, the pH of the sample is about 8 to about 11, or about 9 to about 11. In one embodiment, the pH of the sample is at least about 8, at least about 9, at least about 10, or at least about 11. In another embodiment, the pH of the sample is about 8, about 8.5, about 9, about 9.5, about 10, about 10.5, or about 11.

### Sodium Hydroxide

In one embodiment, sodium hydroxide (NaOH) is added to a recombinant protein sample. In one embodiment, NaOH is added to the sample so that the concentration ofNaOH in the sample is about 0.1 mM to about 100 mM. In one embodiment, the concentration of NaOH in the sample is about 0.1 mM, about 0.5 mM, about 1 mM, about 5 mM, about 10 mM, about 15 mM, about 20 mM, about 25 mM, about 30 mM, about 35 mM, about 40 mM, about 50 mM, about 60 mM, about 70 mM, about 80 mM, about 90 mM, or about 100 mM.

In one embodiment, after addition of NaOH to the sample, the pH of the sample is at least about 8, at least about 9, at least about 10, or at least about 11. In another embodiment, the pH of the sample is about 8 to about 11, or about 9 to about 11. In one embodiment, the pH of the sample is about 8, about 8.5, about 9, about 9.5, about 10, about 10.5, or about 11.

### Heparin

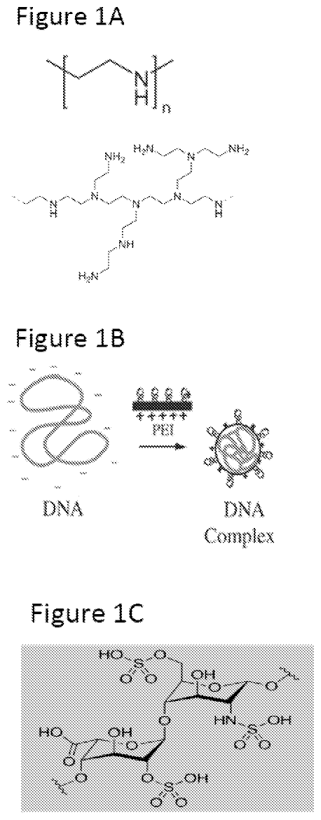

Heparin is a molecule with a strong negative charge. It can be used as an anti-coagulant (blood thinner). The structure of heparin is shown in FIG. 1C.

In one embodiment, heparin is added to a recombinant protein sample. In one embodiment, heparin is added to the sample so that the concentration of heparin in the sample is about 50 μg/mL to about 1000 μg/mL. In another embodiment, the concentration of heparin is about 80 μg/mL to about 750 _{K}g/mL. In one embodiment, the concentration of heparin in the sample is at least about 50 μg/mL, at least about 60 μg/mL, at least about 70 μg/mL, at least about 80 μg/mL, at least about 90 μg/mL, at least about 100 μg/mL, at least about 200 μg/mL, at least about 250 μg/mL, at least about 300 μg/mL, at least about 400 μg/mL, at least about 500 μg/mL, at least about 600 μg/mL, at least about 700 μg/mL, at least about 750 μg/mL, at least about 800 μg/mL, at least about 900 μg/mL, or at least about 1000 μg/mL. In one embodiment, the concentration of heparin in the sample is about 50 μg/mL, about 60 μg/mL, about 70 μg/mL, about 80 μg/mL, about 90 μg/mL, about 100 μg/mL, about 200 μg/mL, about 250 μg/mL, about 300 μg/mL, about 400 μg/mL, about 500 μg/mL, about 600 μg/mL, about 700 μg/mL, about 750 μg/mL, about 800 μg/mL, about 900 μg/mL, or about 1000 μg/mL.

### Flocculant

Flocculants cause the aggregation of insoluble or solid material, such that the soluble recombinant protein remains in solution.

Flocculants include: mineral or vegetable hydrocolloids; cationic polyelectrolytes (for example polyethyleneimine (PEI), cationic polyacrylamide); Poly(diallyldimethylammonium chloride (PDADMAC); polyamines; polyaminoacids; polyacrylamides; polyallylamine; polyvinylamine; poly-N-methylvinylamine (PMVA); natural polymers from microorganisms (for example Chitosan); and chemical flocculants, for example aluminium sulphate, synthetic and non-synthetic polymers. Specific examples of flocculants include PEI, Poly(diallyldimethylammonium chloride) (PDADMAC) (low molecular weight version MW: 100 kDa to 200 kDa; or high molecular weight version 400 kDa to 500 kDa), Acid precipitation, CaCl₂, Chitosan (MW: 110 kDa).

In one embodiment, the flocculant is a cationic polymer. In one embodiment, the flocculant is polydiallyldimethylammonium chloride (pDADMAC), polyamine, polyaminoacid, polyacrylamide, chitosan, polyallylamine, polyvinylamine, polyethyleneimine (PEI), or poly-N-methylvinylamine (PMVA).

In one embodiment, the flocculant is polytheyleneimine (PEI). Polyethyleneimine (PEI) is an organic polycation (positively charged polymer). PEI can be linear, branched (low branch, high branch), and has a strong positive charge (FIG. 1A). PEI can bind DNA strongly and tightly (FIG. 1B). PEI can used as a flocculant to remove host cell DNA and HCP in downstream purification process. PEI can be used with microbial cell cultures and CHO platform downstream purification processes. PEI also is widely used as non-viral vector for gene therapy and transfection. Typically, PEI used in downstream purification is highly branched, with molecular weight of 750 kDa.

In one embodiment, the flocculant is cationic. In one embodiment, the flocculant is polyethyleneimine (PEI). In one embodiment, the PEI is highly branched. In one embodiment, the PEI has a molecular weight of about 750 kDa.

In one embodiment, the recombinant protein sample contains between about 0 to about 1000 ppm flocculant, or about 10 to about 1000 ppm, or about 20 to 1000 ppm, or about 100 to about 1000 ppm, or about 20 to about 200 ppm. In one embodiment, the sample contains at least about 0.001 ppm flocculant, at least about 0.01 ppm flocculant, at least about 0.1 ppm flocculant, at least about 1 ppm flocculant, at least about 10 ppm flocculant, at least about 20 ppm flocculant, at least about 50 ppm flocculant, at least about 100 ppm flocculant, at least about 200 ppm flocculant, at least about 500 ppm flocculant, or at least about 1000 ppm flocculant. In one embodiment, the sample contains about 50 ppm to about 1000 ppm flocculant. In one embodiment, the sample contains about 100 ppm to about 1000 ppm flocculant. In one embodiment, the sample contains about 10 ppm to about 500 ppm flocculant. In one embodiment, the sample contains about 20 ppm to about 200 ppm flocculant.

In one embodiment, the sample contains between about 0 to about 1000 ppm polyethyleneimine (PEI). In one embodiment, the sample contains at least about 0.001 ppm PEI, at least about 0.01 ppm PEI, at least about 0.1 ppm PEI, at least about 1 ppm PEI, at least about 10 ppm PEI, at least about 20 ppm PEI, at least about 50 ppm PEI, at least about 100 ppm PEI, at least about 200 ppm PEI, at least about 500 ppm PEI, or at least about 1000 ppm PEI. In one embodiment, the sample contains about 50 ppm to about 1000 ppm PEI. In one embodiment, the sample contains about 100 ppm to about 1000 ppm PEI. In one embodiment, the sample contains about 10 ppm to about 500 ppm PEI. In one embodiment, the sample contains about 20 ppm to about 200 ppm PEI.

In one embodiment, heparin and sarkosyl are added to a recombinant protein sample so that the sample contains PEI, heparin, and sarkosyl at a ratio of about 1000 ppm PEI:750 μg/mL heparin: 1% sarkosyl.

In one embodiment, SDS and sodium hydroxide (NaOH) are added to the sample, wherein the concentration of SDS in the sample is about 0.5%, and the concentration of NaOH in the sample is about 25 mM.

In one embodiment, sarkosyl and heparin are added to the sample, wherein the concentration of sarkosyl in the sample is about 1.0%, wherein the concentration of heparin in the sample is about 750 μg/mL, wherein the sample comprises about 1000 ppm PEI, and wherein the sample comprises about 1 mg/mL to about 15 mg/mL recombinant protein. In another embodiment, sarkosyl and heparin are added to the sample, wherein the concentration of sarkosyl in the sample is about 0.05%, wherein the concentration of heparin in the sample is about 80 μg/mL, wherein the sample comprises about 100 ppm PEI, and wherein the sample comprises about 1 mg/mL to about 15 mg/mL recombinant protein.

In one embodiment, there is provided a method for detecting host cell DNA in a sample comprising about 1 mg/mL to about 15 mg/mL recombinant protein and about 100 ppm to about 1000 ppm polyethyleneimine (PEI), the method comprising
(a) adding to the sample heparin and sarkosyl, wherein the concentration of heparin is about 80 μg/mL to about 750 μg/mL, and the concentration of sarkosyl is about 0.05% to about 1.0%,
(b) amplifying at least a portion of the host cell DNA, and
(c) detecting the amplification in step (b), thereby detecting the host cell DNA in the sample.

In one embodiment, there is provided a method for detecting host cell DNA in a sample comprising about 1 mg/mL to about 15 mg/mL recombinant protein, the method comprising
(a) adding to the sample heparin and sarkosyl, wherein the concentration of heparin is about 80 μg/mL to about 750 μg/mL, and the concentration of sarkosyl is about 0.05% to about 1.0%,
(b) amplifying at least a portion of the host cell DNA, and
(c) detecting the amplification in step (b), thereby detecting the host cell DNA in the sample.

In one embodiment, there is provided a method for detecting host cell DNA in a sample comprising about 100 mg/mL to about 120 mg/mL recombinant protein and about 20 ppm to about 200 ppm polyethyleneimine (PEI), the method comprising
(a) adding to the sample sodium dodecyl sulfate (SDS) and sodium hydroxide (NaOH), wherein the pH of the sample is at least about 8, wherein the concentration of SDS in the sample is about 0.5%, wherein the concentration of NaOH in the sample is about 25 mM,
(b) amplifying at least a portion of the host cell DNA, and
(c) detecting the amplification in step (b), thereby detecting the host cell DNA in the sample.

In one embodiment, there is provided a method for detecting host cell DNA in a sample comprising about 100 mg/mL to about 120 mg/mL recombinant protein, the method comprising
(a) adding to the sample sodium dodecyl sulfate (SDS) and sodium hydroxide (NaOH), wherein the pH of the sample is at least about 8, wherein the concentration of SDS in the sample is about 0.5%, wherein the concentration of NaOH in the sample is about 25 mM,
(b) amplifying at least a portion of the host cell DNA, and
(c) detecting the amplification in step (b), thereby detecting the host cell DNA in the sample.

In one embodiment, there is provided a method for detecting host cell DNA in a sample comprising about 100 mg/mL to about 120 mg/mL recombinant protein and about 20 ppm to about 200 ppm polyethyleneimine (PEI), the method comprising
(a) adding to the sample sodium dodecyl sulfate (SDS), wherein the concentration of SDS in the sample is about 0.5%,
(b) adjusting the pH of the sample to at least about 8,
(c) amplifying at least a portion of the host cell DNA, and
(d) detecting the amplification in step (b), thereby detecting the host cell DNA in the sample.

In one embodiment, there is provided a method for detecting host cell DNA in a sample comprising about 100 mg/mL to about 120 mg/mL recombinant protein, the method comprising
(a) adding to the sample sodium dodecyl sulfate (SDS), wherein the concentration of SDS in the sample is about 0.5%,
(b) adjusting the pH of the sample to at least about 8,
(c) amplifying at least a portion of the host cell DNA, and (d) detecting the amplification in step (b), thereby detecting the host cell DNA in the sample.

In one embodiment, the concentration of nucleic acid in the sample is about 10⁹ μg/mL to about 10³ μg/mL. In one embodiment, the concentration of nucleic acid in the sample is about 10⁹ μg/mL, about 10⁸ μg/mL, about 10⁷ μg/mL, about 10⁶ μg/mL, about 10⁵ μg/mL, about 10⁴ μg/mL, or, about 10³ μg/mL. In one embodiment, the nucleic acid in the sample is DNA. In one embodiment, the nucleic acid in the sample is host cell DNA.

In one embodiment, the method further comprises the step of denaturing the nucleic acid in the sample. In one embodiment, the step of denaturing the nucleic acid comprises heat denaturation of the nucleic acid. In one embodiment, the step of denaturing the nucleic acid comprises incubating the sample at a temperature of about 85° C., about 90° C., or about 95° C. In one embodiment, the step of denaturing the nucleic acid comprises incubating the sample at about 85° C., about 90° C., or about 95° C. for about 5 minutes, about 10 minutes, or about 15 minutes. In one embodiment, the step of denaturing the nucleic acid is done after the step of adding detergent to the sample. In one embodiment, the step of denaturing the nucleic acid is done after the step of adding detergent and NaOH to the sample. In another embodiment, the step of denaturing the nucleic acid is done after the steps of adding detergent to the sample and adjusting the pH of the sample to at least about 8. In another embodiment, the step of denaturing the nucleic acid is done after the step of adding heparin and detergent to the sample. In one embodiment, the nucleic acid is DNA.

In one embodiment, the method further comprises the step of centrifuging the sample. In one embodiment, the step of centrifuging the sample comprises centrifuging the sample at about 10000 rpm to about 16000 rpm. In one embodiment, the sample is centrifuged at about 10000 rpm, about 11000 rpm, about 12000 rpm, about 13000 rpm, about 14000 rpm, about 15000 rpm, or about 16000 rpm. In one embodiment, the step of centrifuging the sample comprises centrifuging the sample at about 10000 rpm, about 11000 rpm, about 12000 rpm, about 13000 rpm, about 14000 rpm, about 15000 rpm, or about 16000 rpm for about 5 minutes, about 10 minutes, or about 15 minutes. In one embodiment, the step of centrifuging the sample is done after the step of adding detergent to the sample. In one embodiment, the step of centrifuging the sample is done after the step of adding detergent and NaOH to the sample. In another embodiment, the step of centrifuging the sample is done after the steps of adding detergent to the sample and adjusting the pH of the sample to at least about 8. In another embodiment, the step of centrifuging the sample is done after the step of adding heparin and detergent to the sample. In another embodiment, the step of centrifuging the sample is done after the step of denaturing the nucleic acid. In one embodiment, the nucleic acid is DNA.

In one embodiment, a method for detecting a nucleic acid in a sample comprising a recombinant protein and a flocculant is provided, the method comprising
(a) adding a detergent and sodium hydroxide (NaOH) to the sample,
(b) denaturing the nucleic acid,
(c) centrifuging the sample,
(d) amplifying at least a portion of the nucleic acid, and
(e) detecting the amplification in step (d), thereby detecting the nucleic acid in the sample.

In another embodiment, a method for detecting a nucleic acid in a sample comprising a recombinant protein and a flocculant is provided, the method comprising
(a) adding a detergent to the sample,
(b) adjusting the pH of the sample to at least about 8,
(c) denaturing the nucleic acid,
(d) centrifuging the sample,
(e) amplifying at least a portion of the nucleic acid, and
(f) detecting the amplification in step (e), thereby detecting the nucleic acid in the sample.

In still another embodiment, a method for detecting nucleic acid in a sample comprising a flocculant and a recombinant protein is provided, the method comprising
(a) adding heparin and a detergent to the sample,
(b) denaturing the nucleic acid,
(c) centrifuging the sample,
(d) amplifying at least a portion of the nucleic acid, and
(e) detecting the amplification in step (d), thereby detecting the nucleic acid in the sample.

### EXAMPLES

The inventors have devised a sample preparation protocol that has demonstrated the ability to overcome the interference effect due to PEI for residual DNA qPCR assay. The sample preparation developed utilizes a novel mixture of an anionic agent (heparin) and detergent (sarkosyl) added to the samples prior to heat denaturation and centrifugation. Other sample preparations developed utilized a detergent (SDS) and sodium hydroxide added to the samples. In some sample preparations, PEI was separated and removed (either partially or completely) from the samples by separating PEI and DNA with heparin/sarkosyl and centrifuging the sample to separate the PEI/heparin/sarkosyl complex from the DNA, which remained in solution after centrifugation. At least some of the samples were in-process or bulk drug substance samples containing mAb1mAb2, or mAb3. mAb1mAb2, and mAb3, which are monoclonal antibodies, each of which binds a different target.

### Example 1

PEI Interference on qPCR

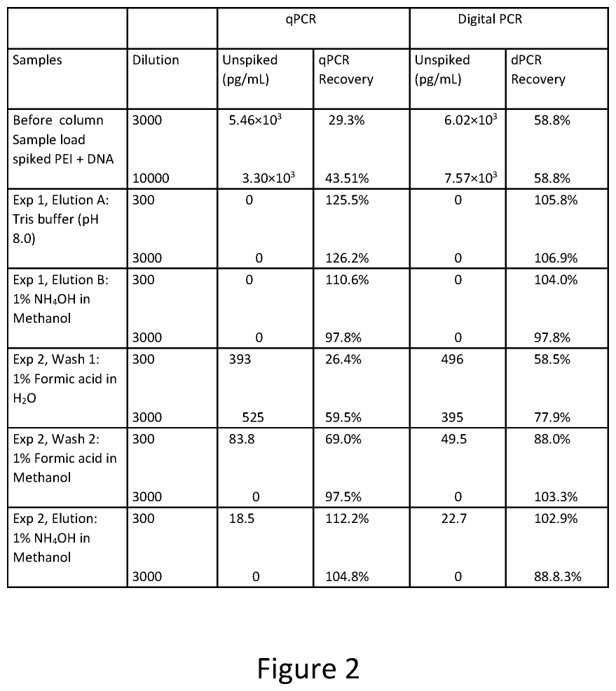

Typically, there is 100 ppm (0.01%) PEI in in-process samples and 20 ppm (0.002%) PEI in BDS (Bulk drug substance) samples. Those samples needed to be diluted 2000-fold to 10000-fold to get acceptable recovery of DNA by qPCR (FIG. 2). This causes two problems. First, there is a decrease in assay sensitivity, as the result is reported as less than the limit of quantification (LOQ)×dilution factor, dilution factor is 10000 and LOQ is 1 pg/mL, result is less than 10000 pg/mL. Second, there is significant risk of not being able to meet the FDA's requirement of residual DNA (10 ng/dose).

Wako DNA/qPCR Results for Samples Treated with Heparin and Sarkosyl

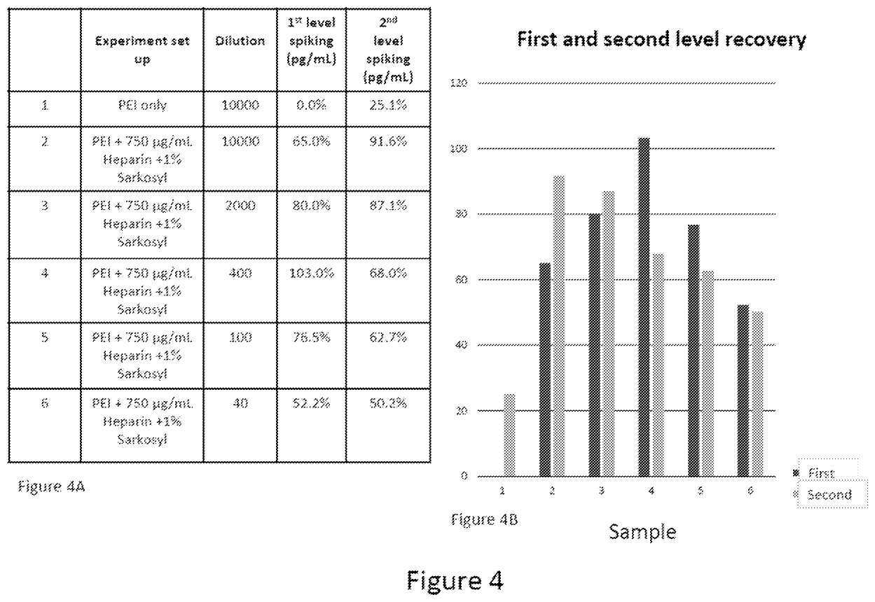

FIG. 4 shows recovery for samples containing PEI 0.1% (1000 ppm)+Chinese hamster ovary (CHO) cell DNA 10⁴ pg/mL final concentration. When PEI only (no treatment) is compared to treatment with heparin and sarkosyl, there is a big difference. With PEI only (no treatment) at 10000 dilution, no DNA was detected by first level spiking, and second level spiking recovery was 25%. With heparin and sarkosyl treatment, at 1:100, first level spiking showed recovery of 76.5%, and second level spiking recovery was 62.7%. Assay sensitivity was improved at least 100-fold.

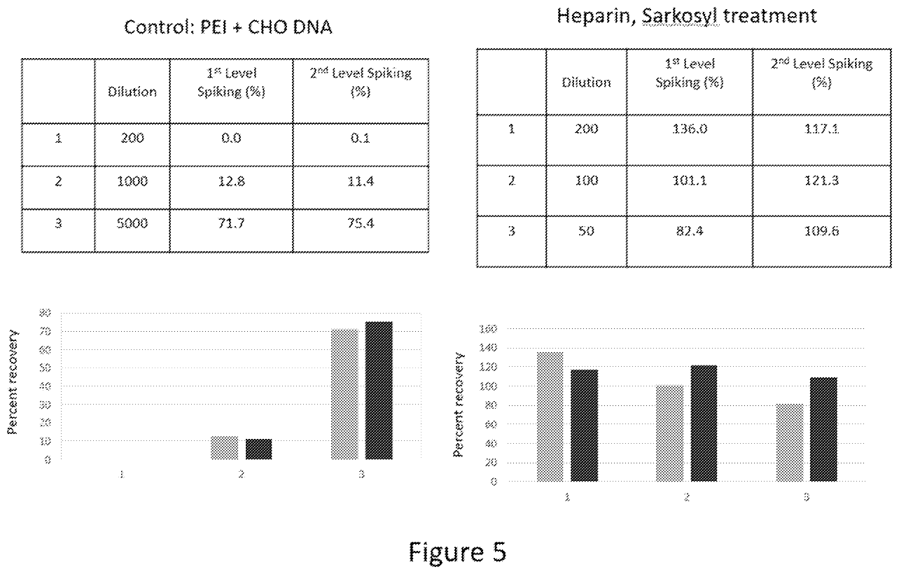

FIG. 5 shows recovery for mAb 1 liquid phenyl eluate samples with 100 ppm PEI treated with heparin (80 μg/mL) and sarkosyl (0.05%).

### Results for Bulk Drug Substance (BDS) Samples

The inventors found that BDS samples treated with heparin and sarkosyl had solid-like consistency, even after heating and centrifuging the samples.

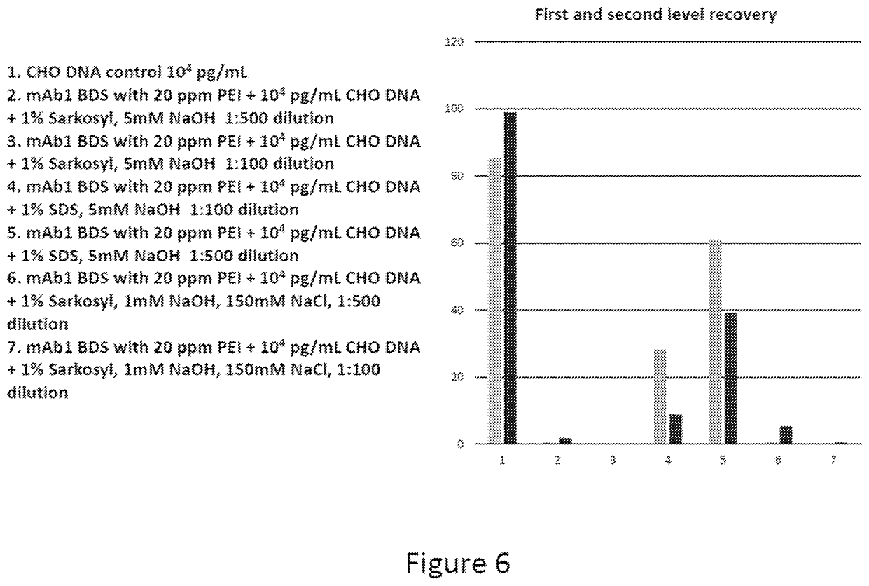

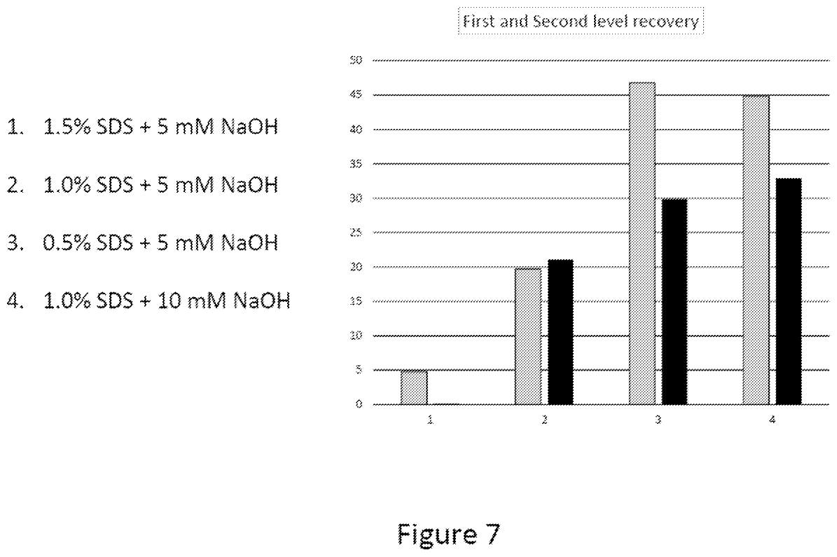

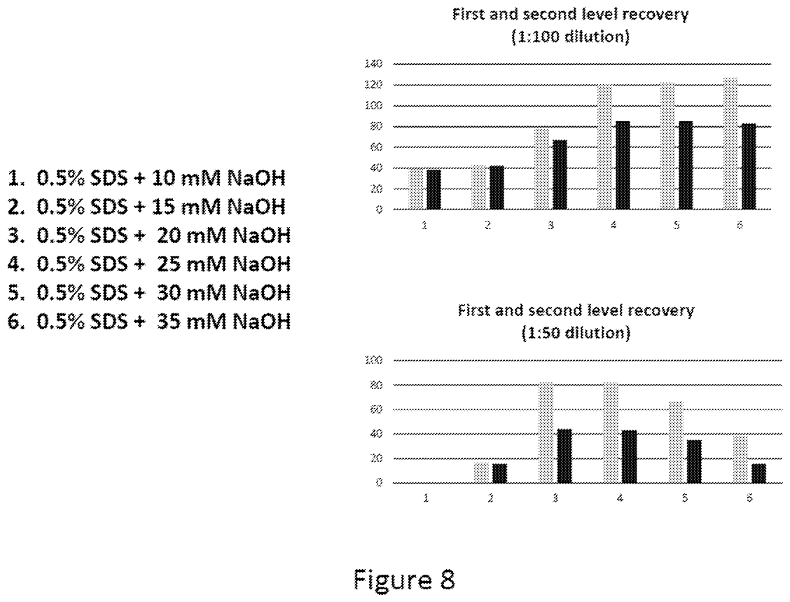

Instead of heparin and sarkosyl, BDS samples were treated with sodium dodecyl sulfate (SDS) and sodium hydroxide (NaOH). FIG. 6 shows recovery for mAb 1 bulk drug substance (BDS) samples with 20 ppm PEI and 10⁴ pg/mL CHO cell DNA, treated with SDS and NaOH. Results in FIG. 6 show that SDS plus NaOH worked better than Sarkosyl. FIGS. 7 and 8 show recovery for mAb2 BDS with 20 ppm PEI and 10⁴ μg/mL CHO cell DNA, treated with SDS and NaOH. The results indicate 0.5% SDS+25 mM NaOH removed PEI from BDS samples. The results in FIG. 7 also show that 0.5% SDS was the best assay condition, and 10 mM NaOH worked better than 5 mM NaOH. Samples treated with SDS and NaOH had a pH of about 9-11.

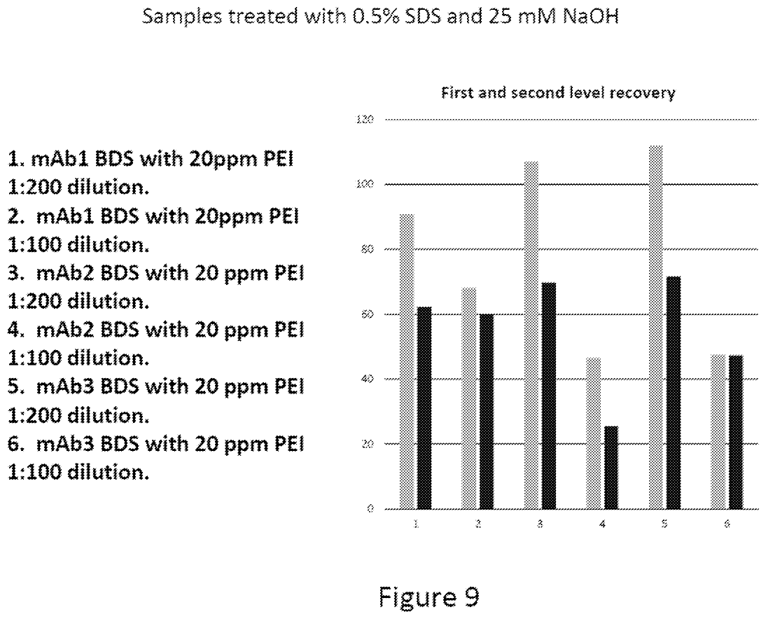

FIG. 9 shows recovery of mAb 1 BDS, mAb2 BDS, and mAb3 BDS samples, with 20 ppm PEI and 10⁴ pg/mL CHO cell DNA, treated with 0.5% SDS and 25 mM NaOH. Without treatment, BDS with 20 ppm PEI needed to be diluted 1:2000 to reach greater than 60% spiking recovery. 0.5% SDS+25 mM NaOH treatment improved the assay sensitivity about 10 times. By 1:200 dilution, qPCR result could easily meet the FDA's requirement of residual DNA qPCR assay.

### Summary of Results

In summary, the results show that using 0.5% SDS and 25 mM NaOH (final concentration) in a sample effectively removed PEI from the sample, and increased assay sensitivity 10-fold for BDS samples (20 ppm PEI). The results also show that 750 μg/mL heparin+1% sarkosyl for 1000 ppm PEI, and 80 μg/mL heparin+0.05% sarkosyl for 100 ppm PEI, increased qPCR assay sensitivity for in-process samples by 20-100 fold.

The results described in Example 1 were obtained using the following materials and methods:

### Materials and Methods

Chemicals and Samples

Heparin sodium salt was purchased from Sigma-Aldrich (H3149). N-Lauroylsarcosine (Sarcosyl) sodium salt was purchased from Sigma-Aldrich (L9150). 10% SDS, Sodium Dodecyl Sulfate Solution was purchased from gibco by Life technology (24730). Sodium hydroxide was purchased from Sigma-Aldrich (S8045). Polyethylenimine 750 kDa (PEI) from Aldrich Chemistry Cat# 181878, Lot# MKBW9508V 50 wt%in H₂O. Sodium deoxycholate from Sigma-Aldrich (D5670). Wako DNA extraction kit from Wako (295-50201) Kingfisher flex automated DNA extraction reagent: EasyMag regents from BioMerieux, EasyMag bufferl #280130, EasyMag buffer2 #280131, EasyMag buffer3 #280132, EasyMag Lysis buffer #280134, EasyMag Magnetic Silica #280133. BDS (Bulk drug substance) and in-process samples of five different antibodies manufactured in CHO cells were used for this study.

CHO DNA qPCR Method

TaqMan Universal PCR Master mix was purchased from Applied Biosystems (Cat# 4304437). 7500 real Time PCR system from Applied Biosystems was used. The primers and probes chosen for this assay target the CHO Alu-2 equivalent sequence. The amplicon size is 107 bp. CHO DNA standard is genomic DNA isolated from CHO DG44 null cell line. DNA concentration was determined by OD 260/280 with spectrophotometer (Agilent 8453). Nuclease-Free Water from Ambion (AM9932). Run TaqMan standard programme 45 cycles, DNA extraction were done in duplicate and qPCR were done by 4 replicate non-spike and 4 replicates spiking. The LOD and LOQ of CHO DNA qPCR are 0.3 and 1.0 pg/mL. Spiking recovery was calculated by mean of concentration of the spiked sample—mean concentration of unspiked sample/10000 pg/mL. The acceptance criteria of spiking recovery is 60%-140%.

Direct Droplet Digital PCR (ddPCR) was also used to compare different assay conditions since Wako DNA extraction/qPCR could be time-consuming and laborious. Bio-Rad ddPCR system (Q×200 Droplet Generator, PX1 PCR plate sealer, T100 Thermal Cycler, QX200 Droplet Reader). ddPCR reagents was purchased form Bio-Rad, ddPCR Suppermix for Probe (Cat#1863010), Droplet Generation Oil for Probe (Cat# 1863005), Droplet Reader Oil (Cat# 1863031). The primers and Probe are the same as what was used by Real Time PCR, CHO DNA standard for copy number per μL to pg/mL conversion is the same as what was used by Real Time PCR.

Sample Preparation

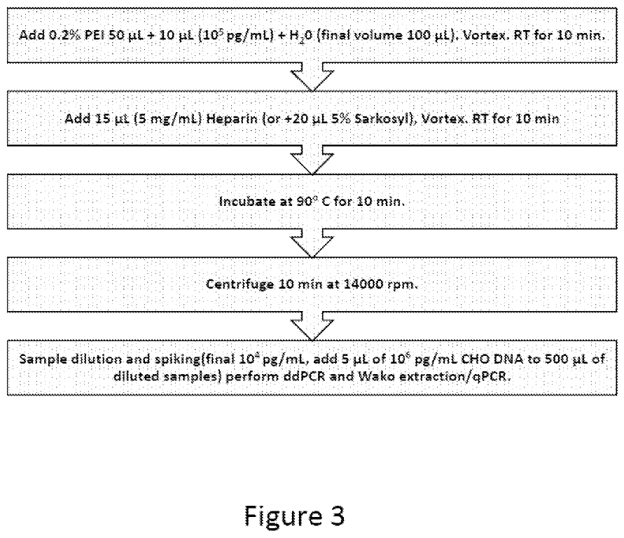

A flow diagram of the sample preparation describe below is shown in FIG. 3.

1.) Add PEI to sample or water, final concentration 20 ppm (0.002%) to 2000 ppm (0.2%). Transfer 50 μL sample or water containing PEI to an Eppendorf tube. Add 10 μL of CHO DNA standard (10⁵ μg/mL or 10⁶ μg/mL). This is first level spiking. Vortex to mix and leave at room temperature for 10 minutes. PEI binds DNA quickly and strongly.

2.) Add Heparin final concentration 80-750 μg/mL, Sarkosyl 0.05-1% final concentration. The total volume is 100 μL. Vortex to mix and leave at room temperature for 10 minutes.

3.) Incubate at 90° C. for 10 minutes by Heatblock.

4.) Centrifuge 10 minutes at 14000 rpm at room temperature.

5.) Sample dilution 1:50-10000 with Nuclease-Free Water, spiking CHO DNA to the samples (final 10⁴pg/mL, add 5μL of 10⁶pg/mL CHO DNA to 500 μL of diluted samples), this is second level spiking. Perform ddPCR or Wako DNA extraction/qPCR.

1. A method for detecting a nucleic acid in a sample comprising a recombinant protein and a flocculant, the method comprising
(a) adding a detergent and sodium hydroxide (NaOH) to the sample,
(b) amplifying at least a portion of the nucleic acid, and
(c) detecting the amplification in step (b), thereby detecting the nucleic acid in the sample.

2. The method of claim 1, wherein the detergent is SDS.

3. The method of claim 1, wherein the flocculant is PEI.

4. The method of claim 1, wherein the sample comprises flocculant at a concentration of about 20 ppm to about 200 ppm.

5. The method of claim 1, wherein the sample comprises recombinant protein at a concentration of about 50 mg/mL to about 250 mg/mL.

6. The method of claim 1, wherein the detergent is SDS, and the concentration of SDS in the sample is about 0.01% to about 2.0%.

7. The method of claim 1, wherein the concentration of NaOH in the sample is about 0.1 mM to about 100 mM.

8. The method of claim 1, wherein the amplification in step (c) comprises a polymerase chain reaction (PCR).

9. The method of claim 1, wherein the nucleic acid is host cell DNA.

10. The method of claim 1, wherein the recombinant protein is an antibody, for example a monoclonal antibody.

11. A method for detecting a nucleic acid in a sample comprising a recombinant protein and a flocculant, the method comprising
(a) adding a detergent to the sample,
(b) adjusting the pH of the sample to at least about 8,
(c) amplifying at least a portion of the nucleic acid, and
(d) detecting the amplification in step (c), thereby detecting the nucleic acid in the sample.

12. The method of claim 11, wherein the detergent is SDS.

13. The method of claim 11, wherein the flocculant is PEI.

14. The method of claim 11, wherein the sample comprises flocculant at a concentration of about 20 ppm to about 200 ppm.

15. The method of claim 11, wherein the sample comprises recombinant protein at a concentration of about 50 mg/mL to about 250 mg/mL.

16. The method of claim 11, wherein the detergent is SDS, and the concentration of SDS in the sample is about 0.1% to about 2.0%.

17. The method of claim 11, wherein the amplification in step (c) comprises a polymerase chain reaction (PCR).

18. The method of claim 11, wherein the nucleic acid is host cell DNA.

19. The method of claim 11, wherein the recombinant protein is an antibody, for example a monoclonal antibody.

20. A method for detecting nucleic acid in a sample comprising a flocculant and a recombinant protein, the method comprising
(a) adding heparin and a detergent to the sample,
(b) amplifying at least a portion of the nucleic acid, and
(c) detecting the amplification in step (b), thereby detecting the nucleic acid in the sample.

21. The method of claim 20, wherein the detergent is sarkosyl.

22. The method of claim 20, wherein the flocculant is PEI.

23. The method of claim 20, wherein the flocculant is present in the sample at a concentration of about 100 ppm to about 1000 ppm.

24. The method of claim 20, wherein the recombinant protein is present in the sample at a concentration of about 0.1 mg/mL to about 20 mg/mL.

25. The method of claim 20, wherein the detergent is sarkosyl and the concentration of sarkosyl in the sample is about 0.01% to about 2.0%.

26. The method of claim 20, wherein the concentration of heparin in the sample is about 50 μg/mL to about 1000 μg/mL.

27. The method of claim 20, wherein the amplification in step (b) comprises a polymerase chain reaction (PCR).

28. The method of claim 20, wherein the nucleic acid is host cell DNA.

29. The method of claim 20, wherein the recombinant protein is an antibody, for example a monoclonal antibody.
